(19) United States
(12) Patent Application Publication
(10) Pub. No.: US 2020/0179673 A1
(43) Pub. Date: Jun. 11, 2020

(54) CONNECTING DEVICE FOR WOUND PROTECTION DRESSING, AND WOUND PROTECTION DRESSING

(71) Applicant: Mianshui WAN, Guangdong (CN)

(72) Inventor: Mianshui WAN, Guangdong (CN)

(21) Appl. No.: 16/614,384

(22) PCT Filed: Jan. 8, 2018
(86) PCT No.: PCT/CN2018/071796

§ 371 Date: Nov. 18, 2019

(51) Int. Cl. A61M 39/10; A61M 1/00; A61F 13/00

(52) U.S. Cl. CPC A61M 39/1055; A61F 13/00068; A61M 1/0088; A61M 1/0058

(57) ABSTRACT

A connecting device for a wound protection dressing, and a wound protection dressing. The connecting device comprises a first tube connector (1) and a second tube connector (3); the first tube connector (1) and the second tube connector (3) are respectively provided with a first through hole (1a) and a second through hole (3a) that run through the respective ends of the first tube connector (1) and the second tube connector (3); a hollow ball head (2) is integrally formed on one end of the first tube connector (1); the inner cavity of the ball head (2) are in communication with the first through hole (1a); at least one via hole (2a) is provided on the ball head (2); a ball shell (4) adapted to the ball head (2) is integrally formed on one end of the second tube connector (3); the inner cavity of the ball shell (4) is in communication with the second through hole (3a); a snap-fit hole (4a) is provided on the ball shell (4); the snap-fit hole (4a) has at least one circular fastening hole (4a1); the ball head (2) is snap-fit in the ball shell (4); the end of the first tube connector (1) close to the ball head (2) is fastened in one of the fastening hole (4a1) of the snap-fit hole (4a). Thus, the connecting device for a wound protection dressing and the wound protection dressing which avoid pressing the skin and wound surface, allow the liquid to smoothly flow out, and facilitate flexible connection of the tube line are provided.

### BACKGROUND OF THE INVENTION

The invention belongs to the field of medical health, and particularly relates to a connecting device for wound dressing and a wound dressing.

In the prior art, a series of wound treatment and wound protection devices are designed by the inventor, and application of the wound wound treatment device is as follows: the application number of the wound wound treatment device is 201110240452.3, a flushing-drainage type wound wound protection device (application number 20122013474.5), a directional irrigation type wound wound protection device (application number 201220134776.9), an independent irrigation-drainage function module wound repair device (application number 20123090002.6), and an active flushing type negative pressure drainage wound protection device (the application number is 20131200447X). The wound wound treatment and wound protection device for manufacturing the sponge structure dressing with the drainage tube and the flushing tube has the advantages that the good effect is achieved clinically.

However, due to the fact that a pipeline of an existing wound protection dressing is a drainage tube or a flushing tube, the wound dressing is led out in a straight line parallel to the skin. The straight pipeline leading-out mode mainly comprises the following defects:

The method comprises the following defects: compressing skin on the periphery of a wound wound by virtue of a pipeline, and causing skin edema and even necrosis;

Secondly, sealing of the periphery of the pipeline is complicated, the situation that closed air leakage occurs easily exist, not only is the wound protection dressing lose drainage effect, but also surrounding sponge water loss can be reduced, and the problem that wound or wound is polluted by bacteria in outside air is solved.

Therefore, the manufacturer adopts the direction perpendicular to the skin to lead out the liquid inlet port of the irrigation pipe and/or the liquid outlet port of the drainage pipe, and specifically, the pipeline and the outside pipeline in the wound protection dressing are respectively in sealed connection with the horizontal joint and the vertical joint of the L-shaped corner joint in an adhesive manner. The L-shaped corner joint is made of medical silica gel or medical rubber. In this way, the problems that the pipeline is complex and unreliable are solved, but the following defects still exist:

Under the action of negative pressure of the drainage tube, the corner joint is easily subjected to compression deformation and then is combined with movement of a human body, the corner joint subjected to compression deformation is easily bent to form a sharp part, the sharp part presses the wound of the wound, and edema and necrosis are caused;

Secondly, after the corner joint is bent to form a sharp part, smooth output of liquid of the drainage tube is influenced, so that the risk of liquid leakage and gas leakage caused by liquid accumulation is avoided, and sealing failure is caused;

The method is troublesome in bonding operation between the pipeline and the corner joint and inconvenient to use.

In addition, in clinical use, for a large-area wound surface, a plurality of wound protection dressing needs to be spliced into a whole which is adapted to the wound surface, so that overall movement and operation in the operation are facilitated. When the wound protection dressing is spliced, if only the sponge is sewn, and the pipelines in each wound dressing are not connected, only a part of negative pressure conduction can be realized between the plurality of wound protection dressings, and the flushing function can not be basically shared; moreover, a plurality of pipelines are led out, so that the surrounding skin is easily pressed, the risk of air leakage is increased, and the treatment effect is reduced. If the internal pipelines of the plurality of wound protection dressings are to be communicated, the pipelines in all the wound protection dressings are adhered, or the original pipeline is taken out, and a new integral pipeline is re-arranged. The method has the advantages that the bonding operation is troublesome, time and labor are wasted, the time and labor are wasted, and the like. In fact, the use convenience and the use effect are comprehensively considered, and the method for suturing the wound dressing sponge in clinic and not connecting the pipelines is generally adopted.

### BRIEF SUMMARY OF THE INVENTION

The technical problem to be solved by the invention is to provide a connecting device for wound protection dressing, so as to overcome the compression problem of a pipeline joint deformation on a wound surface and skin, avoid influence on smooth output of liquid and facilitate flexible splicing of pipelines.

According to the technical scheme, the connecting device for the wound protection dressing is characterized by comprising a first tube joint and a second tube joint, wherein a first through hole penetrating through the two ends of the first tube joint is formed in the first tube joint, and a second through hole penetrating through the two ends of the second tube joint is also formed in the second tube joint; a hollow ball head is integrally formed at one end of the first pipe joint, an inner cavity of the ball head is communicated with a first through hole of the first pipe joint, and at least one through hole is formed in the ball head; a ball shell matched with the ball head is integrally formed at one end of the second pipe joint, an inner cavity of the ball shell is communicated with a second through hole of the second pipe joint, a clamping hole is formed in the ball shell, the clamping hole is provided with at least one round clamping hole, and the ball head is clamped in the ball shell through a clamping hole; the end, close to the ball head, of the first pipe joint is clamped in one of the clamping holes of the clamping hole, so that the first pipe joint and the second pipe joint are clamped together, and the first through hole is communicated with the second through hole.

By the adoption of the structure, the ball head and the ball shell are arranged to be connected with the first pipe joint and the second pipe joint and are communicated with the first through hole and the second through hole, a clamping hole formed by a plurality of clamping holes is formed in the ball shell, and the corresponding end of the first pipe joint is clamped in one clamping hole of the clamping hole; the first tube joint and the ball head are integrally formed by medical silica gel or medical rubber; the second tube joint and the ball shell are integrally formed by medical silica gel or medical rubber; during use, one end, far away from the ball head, of the first tube joint is connected with a drainage tube in the wound protection dressing, and one end, far away from the ball shell, of the second tube joint is connected with an external pipeline to form a drainage channel; The use of such a connecting device for the drainage channel of the wound protection dressing has the following advantages:

The butt joint ball head and the ball shell adopt a double-layer ball body, the strength is higher, and the ball head and the ball shell are not easy to deform under the action of negative pressure;

The spherical structure of the ball head and the spherical shell, so that pressure dispersion is facilitated, and deformation is further avoided;

Thirdly, even if the negative pressure is large, only the ball head is slightly deformed, ball shell protection is arranged outside the ball head, and slight deformation of the ball head cannot compress the wound surface;

Fourth, the outer diameter of one end, close to the ball head, of the first pipe joint is smaller than the outer diameter of the ball head, and the outer diameter of one end, close to the ball shell, of the second pipe joint is smaller than the outer diameter of the ball shell; the corresponding end of the first pipe joint and the corresponding end of the second pipe joint can support the ball head and the ball shell, and transition deformation of the ball head and the ball shell is avoided;

The ball head is provided with at least one through hole, the number and the position of the through hole are set according to actual needs, so that smooth connection of the first through hole and the second through hole is guaranteed, smooth liquid extraction is ensured, and risks of pipe blockage, liquid leakage and air leakage are reduced;

The joint of the ball head and the ball shell is similar to that of the universal joint; after being clamped, the first tube joint can rotate, and the second tube joint can also rotate around the central axis of the first tube joint, so that the use is more flexible and convenient;

The clamping holes in the ball shell are provided with at least one round clamping hole, the number, the position and the selection of the clamping holes are set according to actual requirements, the angle and direction of connection of the first pipe joint and the second pipe joint are adjusted, and the use is more flexible and convenient;

The clamping connection between the ball head and the ball shell is firm and reliable, connection and disassembly are convenient and fast, and flexible splicing of the drainage tube is facilitated; the joint between the ball head and the ball shell is not required to be sealed due to the connection of the ball head and the ball shell.

Preferably, the central axis of at least one clamping hole of the clamping hole is perpendicular to the central axis of the second through hole of the second pipe joint. When the ball head is clamped into the ball shell, and the corresponding end of the first pipe joint is clamped into the clamping hole, the first pipe joint and the second pipe joint can be vertically clamped, the drainage tube can be conveniently led out of the wound protection dressing in the direction perpendicular to the skin, and compression of the skin around the wound surface caused by direct leading-out is avoided.

Preferably, the clamping hole is provided with three circular clamping holes, the central axis of the three clamping holes is perpendicular to the central axis of the second through hole of the second pipe joint, the center shaft of one clamping hole is perpendicular to the central axis of the second through hole of the second pipe joint, the center shaft of one clamping hole and the center shaft of the second through hole of the second pipe joint are located in the same straight line, and the center axis of one clamping hole and the center axis of the second through hole of the second pipe joint form an included angle of 135 degrees. In this way, the clamping holes are formed, so that the connection between the first pipe joint and the second pipe joint can be in straight connection and can also be vertically connected, and can also be connected with a 135-degree included angle.

At least one third pipe joint is further integrally connected to the ball shell, the third pipe joint is provided with a third through hole penetrating through the two ends, and the inner cavity of the ball shell is communicated with the third through hole. In this way, the third pipe joint is arranged on the ball shell, so that the first pipe joint can be simultaneously connected with the second pipe joint and at least one third pipe joint, so that the drainage pipe can be expanded in the wound protection dressing; furthermore, when the large-area wound is used, the multiple wound dressing can be conveniently spliced.

The second through hole in the second pipe joint comprises a first conical section and a second conical section which are communicated in sequence, a first small-diameter section and a first large-diameter section, the inner diameter of the first small-diameter section is smaller than the inner diameter of the first large-diameter section, the inner diameter of the first small-diameter section is smaller than the inner diameter of the first large-diameter section, the first small-diameter section and the first large-diameter section are in stepped surface transition, the middle of the first large-diameter section protrudes inwards to form a plurality of first clamping blocks uniformly distributed in the circumferential direction, and an interval is reserved between every two adjacent first clamping blocks in this way, the shape of the second through hole of the second pipe joint is set, the first conical section is conveniently matched with the matched pipe joint in a matched mode, and butt joint sealing is facilitated; the existing common infusion tube can be directly inserted into the first small-diameter section and sealed butt joint through tight fit of the first small-diameter section and the first small-diameter section; a first clamping block is arranged in the middle of the first large-diameter section and is clamped with a matched pipe joint conveniently, and the part, located on the outer side of the first clamping block, of the first large-diameter section is sealed through a sealing ring on the matched pipe joint.

The invention further comprises a fourth pipe joint which is in butt joint with the second pipe joint, the fourth pipe joint is internally provided with a fourth through hole penetrating through the two ends, the fourth pipe joint comprises a first conical tube and a first circular tube section which are connected in sequence, the outer wall of the first conical tube is matched with the inner wall of the first conical section of the second through hole, the middle of the first circular tube section protrudes outwards to form a sealing ring and a plurality of second clamping blocks uniformly distributed in the circumferential direction, the sealing ring and the second clamping block are arranged at intervals along the central axis direction of the first circular tube section; the second clamping block is arranged at intervals between the first clamping blocks; when the first conical tube of the fourth tube joint extends into the second tube joint until the second clamping block penetrates through the gap between the first clamping blocks, the second tube joint and the fourth tube joint are rotated to enable the second clamping block to be clamped on the inner side of the first clamping block when the first clamping block is clamped between the second clamping block and the sealing ring, the peripheral side wall of the sealing ring is tightly matched and sealed with the inner wall of the first large-diameter section of the second through hole. In this way, a pipe joint matched with the second pipe joint is formed by the fourth pipe joint, sealing butt joint between the fourth pipe joint and the second pipe joint is realized through the first conical section and the first round pipe section, and the pipe joint has the advantages of being simple in structure, easy to produce and convenient to operate.

The first through hole of the first pipe joint is conical, the small head end of the first through hole is communicated with the ball head, and the inner wall of one end, far away from the ball head, of the first through hole protrudes inwards to form a third clamping block which is uniformly distributed in the circumferential direction. The conical inner hole and the third clamping block are arranged, so that reliable clamping connection between the first pipe joint and the drainage tube is facilitated, and connection and disassembly are convenient and rapid.

The invention aims to provide a wound protection dressing which is used for reducing compression of a pipeline to a wound surface.

The technical scheme is as follows: the wound protection dressing comprises a connecting device for protecting wound dressing, a flushing sponge and a drainage sponge, wherein a drainage pipe is inserted into the drainage sponge, the drainage pipe is connected with one end, far away from the ball head, of the first pipe joint, the end, far away from the ball shell, of the second pipe joint is exposed out of the wound protection dressing, and a flushing pipe is inserted into the flushing sponge.

According to the structure, the first pipe joint ball head and the second pipe joint ball head shell are adopted for pipeline butt joint, so that the compression of the joint of the pipeline on the wound surface is reduced, and the healing of the wound surface is facilitated. The included angle between the first pipe joint and the second pipe joint can be flexibly arranged according to the design of the clamping hole of the clamping connection hole, so that the angle and the direction of the drainage tube leading out of the wound protection dressing can be flexibly arranged; if the design is that the drainage tube is not straightly led out, compression of the pipeline on the skin around the wound surface can be further avoided, and healing of the wound surface is further facilitated.

The device further comprises a fifth pipe connector, wherein the fifth pipe connector comprises two inner connectors which are located on the same straight line in the center line, the two inner connectors are conical with the small inner and small diameters, and the inner connector is connected with the flushing pipe; the fifth pipe joint further comprises an outer joint which is perpendicular to the center line of the inner joint, and the center line of the outer joint is perpendicular to the center line of the inner joint, the outer end of the outer joint is exposed out of the wound protection dressing, the inner hole of the outer joint comprises a second conical section, a second small diameter section and a second large diameter section which are communicated in sequence, the small head end of the second conical section is communicated with the inner hole opening of the two inner connectors, the inner diameter of the second conical section large end section is equal to the inner diameter of the second small diameter section, the inner diameter of the second small diameter section is smaller than the inner diameter of the second large diameter section, and the second small diameter section and the second large diameter section are in stepped surface transition in this way, the fifth pipe joint is connected with the flushing pipe, so that the outer joint of the fifth pipe joint is conveniently used for leading out the wound protection dressing in the direction perpendicular to the skin along the direction perpendicular to the skin, so that the wound protection dressing is prevented from being pressed by the straight line, and wound healing is further facilitated; and the fifth pipe joint is provided with two through inner connectors, the drainage tube is expanded by combining the first tube joint and the third tube joint, so that convenience and expansion of the wound protection dressing tube and the drainage tube can be realized, and the wound protection dressing convenient to splice is provided; when the wound dressing is used in a large-area wound, the wound protection dressing is more convenient to splice; after being spliced, the wound protection dressing and drainage function can be shared.

Further comprises a sixth pipe joint which is in butt joint with the outer joint of the fifth pipe joint. The sixth pipe joint comprises a second conical pipe and a second circular pipe section. The outer wall of the second conical pipe is matched with the inner wall of the second conical section of the inner hole of the outer joint of the fifth pipe joint. In this way, the sixth pipe joint matched with the fifth pipe joint is arranged, so that the quick and reliable butt joint of the sixth pipe joint and the fifth pipe joint is facilitated.

Advantageous Effects: The invention connects the first pipe joint and the second pipe joint by connecting the ball head and the ball shell, and connects the first through hole and the second through hole, thereby providing a method for avoiding pressing of the skin and the wound surface, and smoothly flowing out of liquid and flexibly splicing pipe. The connecting device for a wound protection dressing and a wound protection dressing have the advantages of being ingenious in concept, easy to transform, low in modification cost and the like.

### BRIEF DESCRIPTION OF THE DRAWINGS

In order to more clearly illustrate the technical solutions in the embodiments of the present invention or in the prior art, the accompanying drawings required for describing the specific embodiments or the prior art are briefly introduced below. In all drawings, like elements or parts are generally identified by like reference numerals. In the drawings, elements or parts are not necessarily drawn in an actual scale.

FIG. 1 is a schematic structural diagram of a first embodiment.

FIG. 2 is a cross-sectional view of the-an of FIG. 1.

FIG. 3 is a structural schematic view of a second tube joint and a ball shell.

FIG. 4 is an use state of one embodiment of the present invention.

FIG. 5 is another use state of the first embodiment.

FIG. 6 is a schematic structural diagram of a second embodiment.

FIG. 7 shows one use state of the second embodiment of the present invention.

FIG. 8 is a schematic structural diagram of a third embodiment.

FIG. 9 is a schematic view of the internal structure of a fifth pipe joint and a sixth pipe joint.

FIG. 10 is a schematic view of the internal structure of a fifth tube joint.

Reference numerals designate corresponding parts throughout the several views: a first tube joint 1, a first through hole 1a, a third clamping block 1b, a ball head 2, a through hole 2a, a second pipe joint 3, a second through hole 3a, a first conical section 3a1, a first small-diameter section 3a2, a first large-diameter section 3a3, a first clamping block 3b, a spherical shell 4, a clamping hole 4a, a clamping hole 4a1, a fourth pipe joint 5, a second clamping block 5a, a sealing ring 5b, a fourth through hole 5c, a first conical tube 511, a first circular tube section 512, a fifth tube joint 6, an inner joint 611, an outer joint 612, a second conical section 612a, a second small diameter section 612b, a second large diameter section 612c, a sixth tube joint 7, a second conical tube 711, a second circular tube section 712, a clamping ring 713, a drainage sponge 8, a flushing sponge 9, a drainage tube 10, a flushing tube 11 and a third tube joint 12.

### DETAILED DESCRIPTION OF THE INVENTION

The embodiments of the technical solutions of the present invention will be described in detail with reference to the accompanying drawings. The following examples are only used for more clearly illustrating the technical solutions of the present invention, and therefore are only used as examples, but cannot limit the scope of protection of the present invention.

In the description of the application, it is to be understood that, the term “center”, “up” and “down”, “before” and “after”, “left”, “right” and “vertical”, “level”, “top”, “bottom” “inside”, “outside”, “axis”, “radial” and “to” etc. the orientation or position relationship is based on the orientation or positional relationship shown in the drawings, but is intended to facilitate describing the invention and the simplified description, rather than indicating or implying that the device or element is required to have a specific orientation, and is constructed and operated in a specific orientation so as not to understand the limitation of the present invention.

Furthermore, the terms “first,” “second,” and the like are used for purposes of description only and are not to be construed to indicate or imply a relative importance or to implicitly indicate the number of technical features indicated. In the description of the present invention, the meanings of “multiple” are more than two, unless explicitly stated otherwise.

In the present application, the terms “installed,” “connected,” “connected,” “fixed,” and the like should be construed broadly, for example, may be fixedly connected, or may be detachably connected, or integrated; the terms “installed”, “connected”, “connected”, “fixed” and the like can be directly connected or indirectly connected through an intermediate medium, and can be in communication between the two elements or an interaction relationship between the two elements. For a person of ordinary skill in the art, the specific meaning of the terms in the invention can be understood according to specific situations.

### First Embodiment

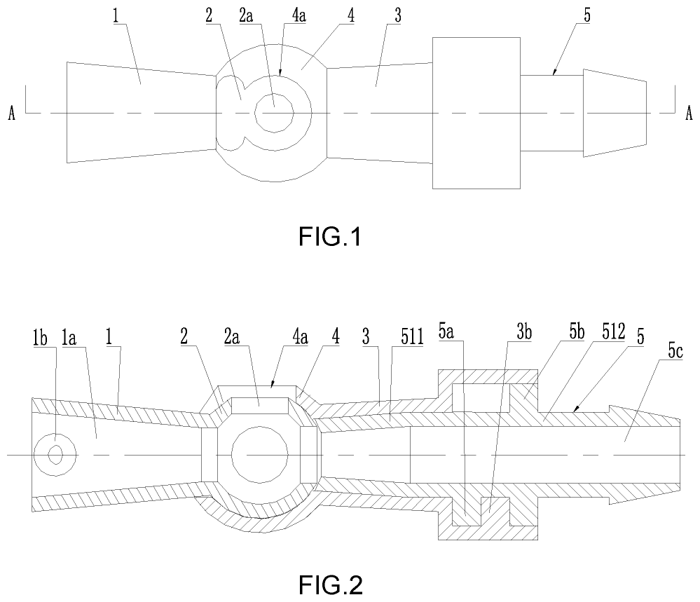

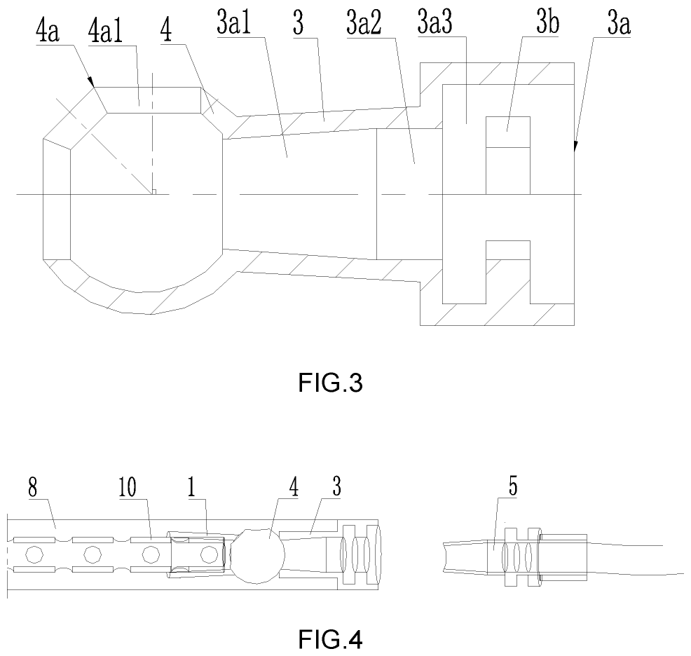

As shown in FIG. 1, as shown in FIG. 2 and FIG. 3, the embodiment of the invention provides a connecting device for a wound dressing, a first tube joint 1 and a second tube joint 3, a first through hole 1a penetrating through the two ends of the first tube joint 1 is formed in the first tube joint 1, a second through hole 3a penetrating through the two ends of the second tube joint 3 is also formed in the second tube joint 3, a hollow ball head 2 is integrally formed at one end of the first tube joint 1, and an inner cavity of the ball head 2 is communicated with the first through hole 1a of the first tube joint 1. In the embodiment, the first tube joint 1 and the ball head 2 are integrally formed by medical silica gel or medical rubber. At least one through hole 2a is formed in the ball head 2, the number and the position of the through hole 2a are not limited, and a person skilled in the art can ensure that the first through hole 1a and the second through hole 3a can be communicated according to actual conditions. In the embodiment, the number of the through holes 2a is four, and the total of the four through holes 2a and the inner hole openings of the first through hole 1a of the first pipe connector 1 are distributed in a front-and-back manner on the ball head 2. The first through hole 1a of the first tube joint 1 is conical, the small head end of the first through hole 1a is communicated with the ball head 2, the inner wall of one end, far away from the ball head 2, of the first through hole 1a protrudes inwards to form a third clamping block 1b which is uniformly distributed in the circumferential direction, and the third clamping block 1b is used for quickly and reliably abutting the drainage tube in the wound protection dressing.

As shown in FIGS. 1, 2 and 3, a ball shell 4 matched with the ball head 2 is integrally formed at one end of the second tube joint 3, and an inner cavity of the ball shell 4 is communicated with a second through hole 3a of the second tube joint 3. In this embodiment, the second tube joint 3 and the ball shell 4 are preferably integrally formed by medical silica gel or medical rubber. A clamping hole 4a is formed in the ball shell 4, the clamping hole 4a is provided with at least one round clamping hole 4a1, the ball head 2 is clamped in the ball shell 4 through the clamping hole 4a, the end, close to the ball head 2, of the first tube joint 1 is clamped in one clamping hole 4a1 of the clamping hole 4a, the first tube joint 1 and the second tube joint 3 are clamped together, and the first through hole 1a and the second through hole 3a pass through the ball head 2 and the ball shell 4 is communicated with the through hole 2a in the ball head 2. The outer diameter of one end, close to the ball head 2, of the first pipe joint 1 is smaller than the outer diameter of the ball head 2, and the outer diameter of the end, close to the ball shell 4, of the second pipe joint 3 is smaller than the outer diameter of the ball shell 4.

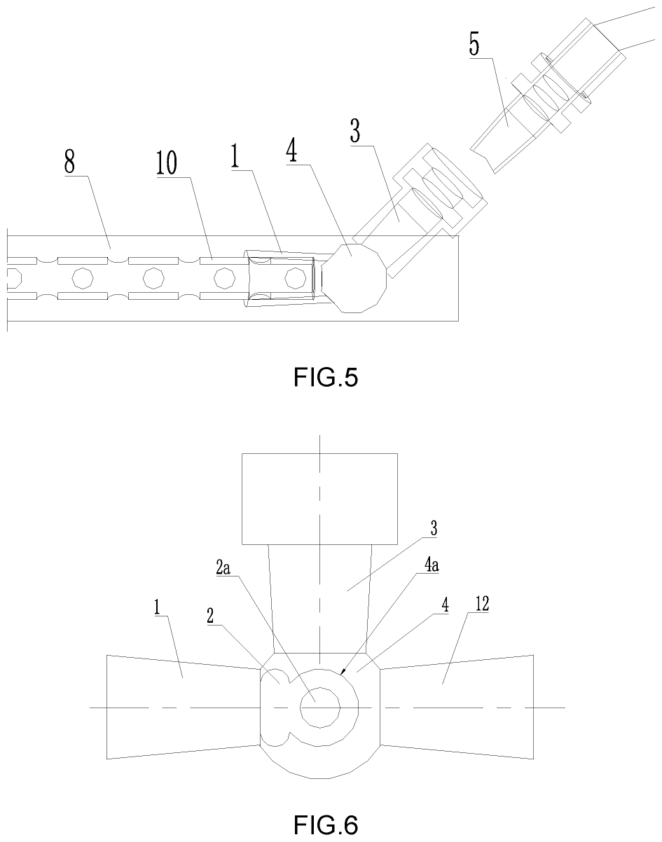

As shown in FIGS. 1, 2 and 3, the central axis of at least one clamping hole 4a1 of the clamping hole 4a is perpendicular to the central axis of the second through hole 3a of the second tube joint 3. Preferably, the clamping hole 4a is provided with three circular clamping holes 4a1 which are in series connection, and the central axis of the three clamping holes 4a1 and the central axis of the second through hole 3a of the second pipe joint 3 are located on the same plane. In addition, the central axis of one of the clamping holes 4a1 is perpendicular to the central axis of the second through hole 3a of the second pipe joint 3, the center axis of one clamping hole 4a1 is located on the same straight line with the center axis of the second through hole 3a of the second pipe joint 3, and the center axis of the remaining clamping hole 4a1 and the center axis of the second through hole 3a of the second pipe joint 3 form an included angle of 135 degrees. As shown in FIG. 4, when the end, close to the ball head 2, of the first tube joint 1 is clamped in the clamping hole 4a1 in the same straight line as the center shaft of the first tube joint 1 and the center shaft of the second through hole 3a, the drainage tube 10 is guided out of the drainage sponge 8 of the wound protection dressing in a straight line as shown in FIG. 5, when the end, close to the ball head 2, of the first tube connector 1 is clamped in the clamping hole 4a1 with the included angle of 135 degrees with the central axis of the central shaft and the central axis of the second through hole 3a, the drainage tube 10 is led out along the 45-degree included angle between the drainage tube 10 and the wound protection dressing.

As shown in FIG. 2 and FIG. 3, the second through hole 3a in the second pipe joint 3 comprises a first conical section 3a1 which is communicated in sequence, the small head end of the first conical section 3a1 is communicated with the ball shell 4, the inner diameter of the large end of the first conical section 3a1 is the same as the inner diameter of the first small diameter section 3a2, the inner diameter of the first small diameter section 3a2 is smaller than the inner diameter of the first large diameter section 3a3, and the first small diameter section 3a2 and the first large diameter section 3a3 are in stepped surface transition. The middle of the first large-diameter section 3a3 protrudes inwards to form a plurality of first clamping blocks 3b which are uniformly distributed in the circumferential direction, and an interval is reserved between every two adjacent first clamping blocks 3b an annular pasting part (not shown) is formed in the peripheral direction of the outer end of the second pipe joint 3 when the central line of the second pipe joint 3 is arranged in the direction perpendicular to the upper surface of the wound protection dressing, the second pipe joint 3 is buried in the drainage sponge 8 of the wound protection dressing, the upper end opening of the second pipe joint 3 is flush with the upper surface of the wound protection dressing, and the top surface of the annular pasting part is flush with the upper surface of the wound protection dressing. In this way, the pasting film is adhered and fixed with the annular pasting part of the second pipe joint 3 to ensure that the sealing is not breathable and does not leak liquid; the outer connecting pipe only needs to pierce the pasting film just facing the upper hole of the second pipe joint 3, so that the pipeline connection can be carried out.

As shown in FIG. 1 and FIG. 2, a fourth pipe joint 5 that interfaces with the second pipe joint 3 is further provided, and the fourth pipe joint 5 has a fourth through hole 5c penetrating both ends. The fourth pipe joint 5 comprises a first conical tube 511 and a first circular tube section 512 which are connected in sequence, the outer wall of the first conical tube 511 is matched with the inner wall of the first conical section 3a1 of the second through hole 3a, and the small head end of the first conical tube 511 is in a crescent shape and is convenient for puncturing the pasting membrane. The middle of the first circular tube section 512 protrudes outwards to form a sealing ring 5b and a plurality of second clamping blocks 5a which are uniformly distributed in the circumferential direction; the sealing ring 5b and the second clamping block 5a are arranged at intervals along the central axis direction of the first circular tube section 512; the second clamping block 5a is arranged at an interval between the first clamping block 3b, when the first conical tube 511 of the fourth tube joint 5 extends into the second tube joint 3 until the second clamping block 5a passes through the gap between the first clamping block 3b, so that the second clamping block 5a is clamped on the inner side of the first clamping block 3b; when the first clamping block 3b is clamped between the second clamping block 5a and the sealing ring 5b, the circumferential side wall of the sealing ring 5b is tightly matched and sealed with the inner wall of the first large-diameter section 3a3 of the second through hole 3a.

### Embodiment 2

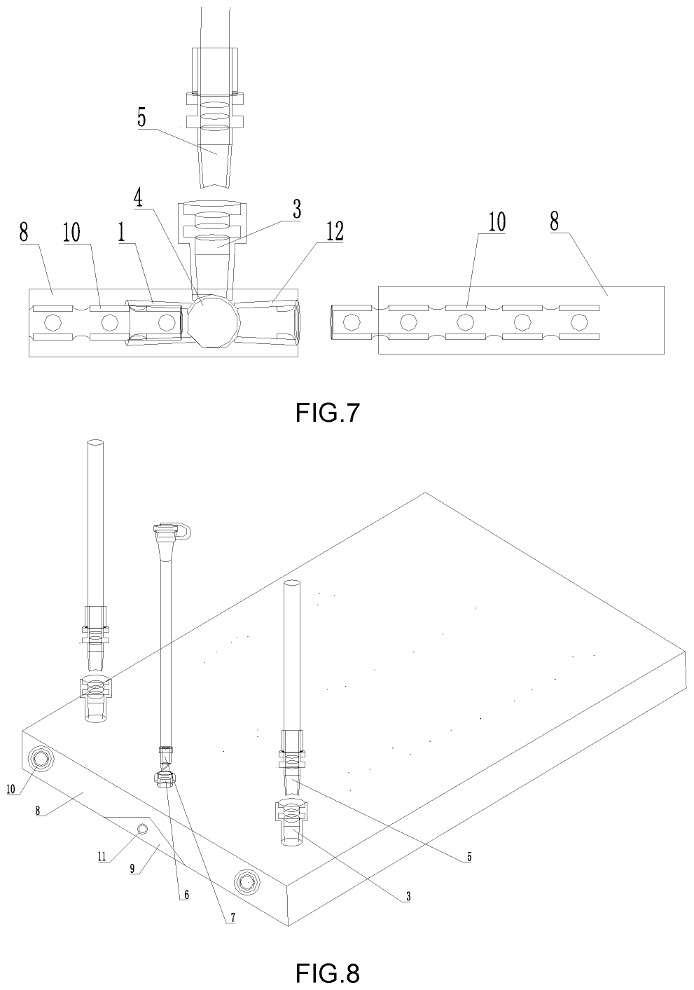

As shown in FIG. 6, at least one third pipe joint 12 is further integrally connected to the ball shell 4, the third pipe joint 12 is provided with a third through hole (not shown) penetrating through the two ends, and the inner cavity of the ball shell 4 is communicated with the third through hole. Preferably, the structure of the third pipe joint 12 is the same as the structure of the first pipe joint 1, the center line of the third pipe joint 12 third through hole is located on the same straight line with the center line of the first through hole 1a of the first pipe joint 1, and the center line of the second pipe joint 3 second through hole 3a is perpendicular to the center line of the first through hole 1a and the center line of the third through hole. Other structures of the embodiment are the same as that of the first embodiment, and are not described in detail herein. As shown in FIG. 7, the third tube connector 12 can be used for being in butt joint with the drainage tube 10 in the adjacent wound protection dressing drainage sponge 8, so that the plurality of wound protection dressing drainage tubes 10 are spliced.

### Embodiment 3

As shown in FIG. 8, the embodiment provides a wound protection dressing, comprising the connecting device for wound protection dressing according to the first embodiment or the second embodiment, further comprising a rinsing sponge 9 and a drainage sponge 8, a drainage tube 10 is inserted into the drainage sponge 8, the drainage tube 10 is connected with one end, far away from the ball head 2, of the first tube joint 1, the end, far away from the ball shell 4, of the second tube joint 3 is exposed out of the wound protection dressing, and the fourth tube joint 5 is located outside the wound protection dressing. A flushing pipe 11 is inserted into the flushing sponge 9.

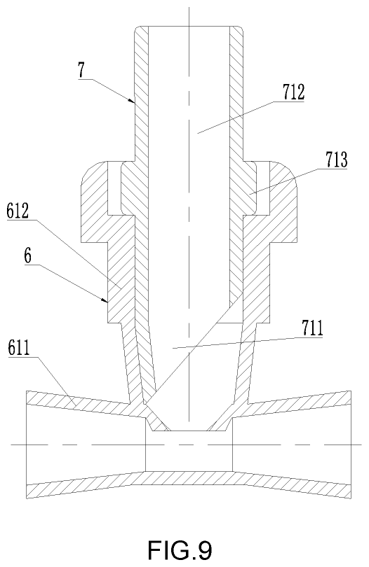

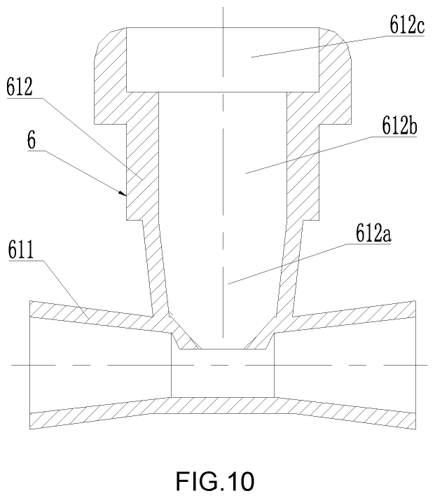

As shown in FIG. 8, FIG. 9 and FIG. 10, the embodiment further comprises a fifth pipe joint 6, the fifth pipe joint 6 comprises two inner joints 611 located on the same straight line in the center line, each inner joint 611 is in a conical shape with an inner small outer part, and the inner joint 611 is connected with the washing pipe 11. The fifth tube joint 6 further comprises an outer joint 612, wherein the center line of the outer joint 612 is perpendicular to the center line of the inner joint 611, and the outer end of the outer joint 612 is exposed out of the wound protection dressing in order to facilitate sealing and pasting of the pasting film, the outer end of the outer joint 612 extends in the circumferential direction to form an annular pasting ring (not shown) the inner hole of the outer joint 612 comprises a second conical section 612a which is communicated in sequence, a second small diameter section 612b and a second large diameter section 612c, the inner diameter of the second conical section 612a is equal to the inner diameter of the second small diameter section 612b, the inner diameter of the second small diameter section 612b is smaller than the inner diameter of the second large diameter section 612c, and the second small diameter section 612b and the second large diameter section 612c are in step surface transition.

As shown in FIGS. 8 and 9, the embodiment further comprises a sixth tube joint 7 which is in butt joint with the outer joint 612 of the fifth tube joint 6, and the sixth tube joint 7 is located outside the wound protection dressing. The sixth pipe joint 7 comprises a second conical tube 711 and a second circular tube section 712, the outer wall of the second conical tube 711 is matched with the inner wall of the second conical section 612a of the inner hole of the outer joint 612 of the fifth tube joint 6, and the small head end of the second conical tube 711 is obliquely sharpened to facilitate puncturing of the pasting membrane. A clamping ring 713 which is matched with the step surface of the inner hole of the outer joint 612 of the fifth pipe joint 6 is formed in the circumferential direction of the outer wall of the second round pipe section 712.

Finally, it should be noted that the above embodiments are only used to illustrate the technical solutions of the present invention and are not limited thereto; although the present invention has been described in detail with reference to the foregoing embodiments, it should be understood by those of ordinary skill in the art that modifications may be made to the technical solutions described in the foregoing embodiments, or equivalent replacements can be made to some or all of the technical features thereof, and these modifications or replacements do not make the essence of the corresponding technical solutions depart from the scope of the technical solutions of the embodiments of the present invention.

1. The connecting device for the wound protection dressing is characterized by comprising a first tube joint and a second tube joint, wherein a first through hole penetrating through the two ends of the first tube joint is formed in the first tube joint, and a second through hole penetrating through the two ends of the second tube joint is also formed in the second tube joint; a hollow ball head is integrally formed at one end of the first pipe joint, an inner cavity of the ball head is communicated with a first through hole of the first pipe joint, and at least one through hole is formed in the ball head; a ball shell matched with the ball head is integrally formed at one end of the second pipe joint, an inner cavity of the ball shell is communicated with a second through hole of the second pipe joint, a clamping hole is formed in the ball shell, the clamping hole is provided with at least one round clamping hole, and the ball head is clamped in the ball shell through a clamping hole; the end, close to the ball head, of the first pipe joint is clamped in one of the clamping holes of the clamping hole, so that the first pipe joint and the second pipe joint are clamped together, and the first through hole is communicated with the second through hole.

2. The connection device for a wound protection dressing according to claim 1, wherein the central axis of the at least one clamping hole of the clamping hole is perpendicular to the central axis of the second through hole of the second pipe joint.

3. The connection device for the wound protection dressing according to claim 2, and is characterized: the clamping hole is provided with three circular clamping holes, the central shaft of the three clamping holes is perpendicular to the central axis of the second through hole of the second pipe joint, the center shaft of one clamping hole is perpendicular to the central axis of the second through hole of the second pipe joint, the center shaft of one clamping hole and the center shaft of the second through hole of the second pipe joint are located on the same straight line, and the center axis of one clamping hole and the center axis of the second through hole of the second pipe joint form an included angle of 135 degrees.

4. The connection device for the wound protection dressing according to claim 1 or 2 or 3 is characterized in that at least one third pipe joint is further integrally connected to the ball shell, the third pipe joint is provided with a third through hole penetrating through two ends, and the inner cavity of the ball shell is communicated with a third through hole.

5. The connection device for the wound protection dressing according to claim 4, and is characterized: the second through hole in the second pipe joint comprises a first conical section and a second conical section which are communicated in sequence, a first small-diameter section and a first large-diameter section, the inner diameter of the first small-diameter section is smaller than the inner diameter of the first large-diameter section, the inner diameter of the first small-diameter section is smaller than the inner diameter of the first large-diameter section, the first small-diameter section and the first large-diameter section are in stepped surface transition, the middle of the first large-diameter section protrudes inwards to form a plurality of first clamping blocks uniformly distributed in the circumferential direction, and an interval is reserved between every two adjacent first clamping blocks.

6. The connection device for the wound protection dressing according to claim 5, and is characterized: and further comprises a fourth pipe joint which is in butt joint with the second pipe joint, the fourth pipe joint is internally provided with a fourth through hole penetrating through the two ends, the fourth pipe joint comprises a first conical tube and a first circular tube section which are connected in sequence, the outer wall of the first conical tube is matched with the inner wall of the first conical section of the second through hole, the middle of the first circular tube section protrudes outwards to form a sealing ring and a plurality of second clamping blocks uniformly distributed in the circumferential direction, the sealing ring and the second clamping block are arranged at intervals along the central axis direction of the first circular tube section, the second clamping block is arranged at intervals between the first clamping blocks, and when the first conical tube of the fourth tube joint extends into the second tube joint until the second clamping block penetrates through the interval between the first clamping blocks; the second clamping block is clamped on the inner side of the first clamping block; when the first clamping block is clamped between the second clamping block and the sealing ring, the circumferential side wall of the sealing ring is tightly matched and sealed with the inner wall of the first large-diameter section of the second through hole.

7. The connection device for the wound protection dressing according to claim 6, characterized in that the first through hole of the first pipe joint is conical, the small head end of the first through hole is communicated with the ball head, and the inner wall of one end, far away from the ball head, of the first through hole protrudes inwards to form a third clamping block uniformly distributed in the circumferential direction.

8. The wound protection dressing is characterized by comprising the connecting device for the wound protection dressing according to claim 1, further comprises flushing sponge and drainage sponge, wherein a drainage pipe is inserted into the drainage sponge, the drainage pipe is connected with one end, far away from the ball head, of the first pipe joint, the end, far away from the ball shell, of the second pipe joint is exposed out of the wound protection dressing, and a flushing pipe is inserted into the flushing sponge.

9. The wound protection dressing according to claim 8, and is characterized: and further comprises a fifth pipe joint, and the fifth pipe joint comprises two inner connectors which are located on the same straight line in the center line, the two inner connectors are conical, the inner joint is connected with the flushing pipe, the fifth pipe joint further comprises an outer joint which is perpendicular to the center line of the inner joint, and the center line of the outer joint is perpendicular to the center line of the inner joint, the outer end of the outer joint is exposed out of the wound protection dressing, the inner hole of the outer joint comprises a second conical section, a second small diameter section and a second large diameter section which are communicated in sequence, the small head end of the second conical section is communicated with the inner hole opening of the two inner connectors, the inner diameter of the second conical section large end section is equal to the inner diameter of the second small diameter section, the inner diameter of the second small diameter section is smaller than the inner diameter of the second large diameter section, and the second small diameter section and the second large diameter section are in stepped surface transition.

10. The wound protection dressing according to claim 9, wherein the sixth pipe joint comprises a second conical pipe and a second circular pipe section; the outer wall of the second conical pipe is matched with the inner wall of the second conical section of the inner hole of the outer joint of the fifth pipe joint; a clamping ring matched with the step surface of the inner hole of the outer joint of the fifth pipe joint is formed in the circumferential direction of the outer wall of the second circular pipe section.
